(19) United States
(12) Patent Application Publication
(10) Pub. No.: US 2024/0318111 A1
(43) Pub. Date: Sep. 26, 2024

(54) NANOFIBER NETWORKS AS MEMBRANE MIMICS FOR IN VITRO APPLICATIONS

(71) Applicant: VIRGINIA TECH INTELLECTUAL PROPERTIES, INC., Blacksburg, VA (US)

(72) Inventors: Philip Graybill, Blacksburg, VA (US); Rafael Davalos, Blacksburg, VA (US); Aniket Jana, Silver Spring, MD (US); Amrinder Nain, Christianburg, VA (US)

(21) Appl. No.: 18/573,665

(22) PCT Filed: Jun. 25, 2022
(86) PCT No.: PCT/US2022/073174

§ 371 Date: Dec. 22, 2023

Related U.S. Application Data
(60) Provisional application No. 63/215,138, filed on Jun. 25, 2021.

(51) Int. Cl. C12M 1/12; C12M 3/06

(52) U.S. Cl. CPC C12M 25/04; C12M 23/16

(57) ABSTRACT

Various examples are provided related to nanofiber networks that can mimic in vivo physiology. In one example, a scaffold includes a nanofiber membrane extending across a scaffold opening. The nanofiber membrane can include a stack of nanofiber layers that include nanofibers disposed with a controlled orientation or direction and a controlled spacing. The nanofibers are cross-linked with nanofibers of an adjacent nanofiber layer. The scaffold can be incorporated into a transwell insert or plate. In another example, a microfluidic chip can include a first channel layer with a first fluid channel; a second channel layer comprising a second fluid channel; and a scaffold including a nanofiber membrane disposed between the first and second channel layers. The first and second channels cross each other.

### CROSS REFERENCE TO RELATED APPLICATIONS

This application claims priority to, and the benefit of, co-pending U.S. provisional application entitled “Nanofiber Networks as Membranes Mimics for In Vitro Applications” having Ser. No. 63/215,138, filed Jun. 25, 2021, which is hereby incorporated by reference in its entirety.

### STATEMENT REGARDING FEDERALLY SPONSORED RESEARCH OR DEVELOPMENT

This invention was made with government support under Grant No. 5P01CA207206-04 awarded by the National Institutes of Health. The government has certain rights in the invention.

### BACKGROUND

Physiological barriers are found throughout the body, and their integrity is key for maintaining healthy functions. These barriers, established by monolayers of endothelial and epithelial cells, restrict transport to enable different microenvironments on either side of the monolayer. Important physiological barriers include the alveolar-capillary barrier, intestinal mucosal barrier, skin barrier, renal epithelial barrier, and the blood-brain barrier (BBB). Barrier properties are derived from junctional complexes between adjacent cells that regulate transport between cells (paracellular transport), and from specialized active and passive transporter proteins that regulate transport across the cell membrane (transcellular transport). Dysfunction of barriers can have significant consequents and have been implicated for a many serious conditions, such as Alzheimer's disease, irritable bowel syndrome, pulmonary edema, and chronic kidney disease, and Atopic dermatitis. Physiological barriers also often present significant blockades for therapeutic agents. Limited drug permeability across these barriers renders many promising drugs/therapies ineffective. For example, nearly 100% of large drugs and 98% for small drugs cannot penetrate the BBB. Barrier properties are also key for immune cell trafficking, cancer metastasis, nanoparticle transport, and other processes.

Although barrier properties are maintained by endothelial and epithelial monolayers, another key aspect of physiological barriers is the basement membrane (BM). The basement membrane is a thin layer of extracellular matrix (ECM) that provides support for endothelial and epithelial cells, while also separating these cells from supporting cell types. The basement membrane in vivo is about 50-100 nm thick and composed of fibrous extracellular (ECM) matrix proteins such as collagen IV, fibronectin, and laminin. Due to the thin and fibrous nature of the BM, endothelial and epithelial cells are regulated by cell-cell crosstalk from cell types opposite the basement membrane. The specialized endothelial cells that comprise the blood-brain barrier, for example, gain their unique transport-restricting properties close contact with adjacent cell types across the basement membrane. FIG. 1 illustrates an example of a BBB formed by specialized endothelial cells supported by pericytes and astrocyte endfeet across a 50-100 nm thick basement membrane. In vivo and in vitro studies have shown that pericytes and astrocytes can regulate endothelial barrier function. In vivo, brain endothelial cells make direct contact with pericytes through peg and socket connections that cross the BM. Astrocyte endfeet surround capillaries in the brain and regulate endothelial behavior through the release soluble factors such as VEGF, TGF β, GDNF, and bFGF. Specifically, supporting cell types such as pericytes and astrocytes can upregulate endothelial tight-junction expression to create tighter barriers. Together, endothelial cells, astrocytes, pericytes, and neurons form what is known as the neurovascular unit (NVU).

### SUMMARY

Aspects of the present disclosure are related to nanofiber networks that can mimic in vivo physiology. In one aspect, among others, a microfluidic chip comprises a first channel layer comprising a first fluid channel; a second channel layer comprising a second fluid channel configured to cross the first fluid channel; and a scaffold disposed between the first and second channel layers, the scaffold comprising a nanofiber membrane separating the first and second fluid channels, the nanofiber membrane comprising a stack of nanofiber layers, each nanofiber layer comprising nanofibers disposed with a controlled orientation and with a controlled spacing, the nanofibers of each nanofiber layer cross-linked with nanofibers of an adjacent nanofiber layer and oriented at an angle with respect to the nanofibers of the adjacent nanofiber layer, the angle in a range between zero and 90 degrees. In one or more aspects, the stack of nanofiber layers can comprise two or more nanofiber layers. The nanofiber membrane can have a thickness from about 5 μm or less. The nanofiber membrane can have an average pore size in a range from about 0.1 μm to about 15 μm. The nanofibers can have a diameter of about 100 nm or larger. The nanofibers can have a diameter in a range from about 200 nm to about 6000 nm.

In various aspects, the nanofibers of each nanofiber layer can be substantially orthogonal to the nanofibers of the adjacent nanofiber layer. The nanofiber membrane can have porosity in a range from about 50% to about 90%. The first and second fluid channels can be substantially orthogonal to each other. The nanofiber membrane separating the first and second fluid channels can have an area of about 3 cm² or greater. The first and second channel layers can comprise polydimethylsiloxane (PDMS). In some aspects, the first channel layer can comprise at least one access channel extending from a surface of the first channel layer to the first fluid channel and the second channel layer can comprise at least one access channel extending from a surface of the second channel layer to the second fluid channel. The at least one access channel of the second channel layer can align with at least one corresponding access channel extending through the first channel layer.

In another aspect, a scaffold comprises a scaffold frame comprising a scaffold opening passing through the scaffold frame; and a nanofiber membrane extending across the scaffold opening, the nanofiber membrane comprising a stack of nanofiber layers, each nanofiber layer comprising nanofibers disposed in a defined direction with a controlled spacing between nanofibers, the nanofibers of each nanofiber layer extending across and cross-linked with nanofibers of an adjacent nanofiber layer. In one or more aspects, the scaffold opening can have an area of about 3 cm² or greater. The nanofibers can have a diameter of about 100 nm or larger. The stack of nanofiber layers can comprise two or more nanofiber layers. The nanofibers of each nanofiber layer can be substantially orthogonal to the nanofibers of the adjacent nanofiber layer.

In various aspects, the nanofiber membrane can have porosity in a range from about 50% to about 90%. The scaffold can be incorporated into a transwell insert for insertion in a well or chamber. One or both sides of the nanofiber membrane can be seeded with one or more cell type. One or both sides of the nanofiber membrane can be seeded with cell-laden hydrogel or extracellular matrix (ECM). The nanofiber membrane can be coated with an attachment factor. The attachment factor can be extracellular matrix (ECM).

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 illustrates an example of a blood-brain barrier formed by specialized endothelial cells supported by pericytes and astrocyte endfeet, in accordance with various embodiments of the present disclosure.

FIG. 2 is a table illustrating examples of membrane mimics used for in vitro modeling, in accordance with various embodiments of the present disclosure.

FIGS. 3A-3C illustrate an example of a microfluidic brain-blood-barrier (BBB) chip, in accordance with various embodiments of the present disclosure.

FIGS. 4A and 4B illustrate an example of polystyrene network fabrication, in accordance with various embodiments of the present disclosure.

FIGS. 5A-5G illustrate a comparison of pore size and distribution in track etched and nanofiber membranes, in accordance with various embodiments of the present disclosure.

FIGS. 6A and 6B illustrate examples of trans-membrane diffusion, in accordance with various embodiments of the present disclosure.

FIGS. 7A-7E illustrate examples of endothelial monolayer formation on nanofiber membranes, in accordance with various embodiments of the present disclosure.

FIGS. 9A-9C illustrate examples of cell-cell contact co-cultures, in accordance with various embodiments of the present disclosure.

FIGS. 10A-10C illustrate an example of a modified transwell plate comprising a nanofiber scaffold, in accordance with various embodiments of the present disclosure.

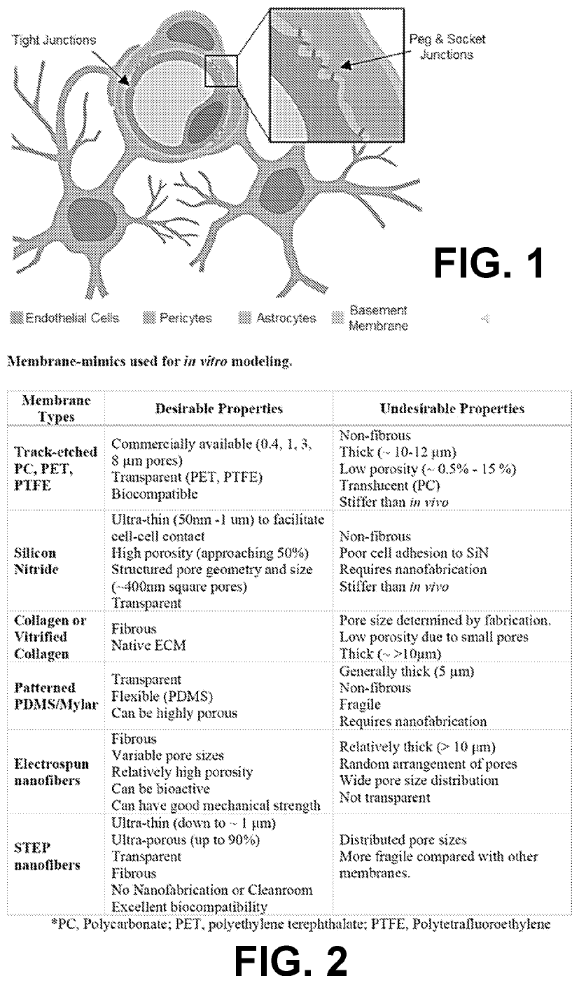

### DETAILED DESCRIPTION

Current cell culture inserts used for modeling the basement-membrane in vitro do not faithfully recapitulate key in vivo physiological properties such as basement membrane thickness, porosity, stiffness, and fibrous composition. Disclosed herein are various examples related to nanofiber networks that can mimic in vivo physiology. Nanofiber networks are described herein, as a new alternative to commercially available cell culture insert membranes for in vitro basement membrane modeling and other applications.

Nanofiber networks, manufactured via the Spinneret-based Tunable Engineered Parameters (STEP) method, use precisely arranged nanofibers to form physiologically relevant basement membranes that closely mimic in vivo physiology. The nanofiber membranes are ultra-thin (e.g., 1 μm-3.2 μm thick), ultra-porous (up to nearly 90% porosity), bio-compatible, and fibrous. Cell-cell contact co-cultures can be established on these membranes for in vitro barrier modeling, such as for blood-brain barrier modeling. These nanofiber networks have impactions for drug discovery, cell migration studies, barrier dysfunction modeling, and other applications.

The nanofiber networks described herein enable close contact between endothelial monolayers and supporting cell types, such as pericytes and astrocytes, which are known to regulate barrier tightness through signaling. Cytoskeletal staining reveals barrier formation on the nanofiber membranes. The nanofiber networks can improve barrier modelling with implications for drug discovery, cell migration studies, and barrier dysfunction modeling. Reference will now be made in detail to the description of the embodiments as illustrated in the drawings, wherein like reference numbers indicate like parts throughout the several views.

Due of the importance of physiological barriers, many in vitro barrier models have been developed for investigating barrier properties. These models rely on endothelial or epithelial monolayers cultured on engineered basement membrane constructs that are designed to mimic the in vivo basement membrane. Although numerous basement-membrane mimicking materials have been developed, to date these membranes still have significant limitations. The ideal basement membrane model would have the following properties: thickness of about 100 nm, fibrous construction, high porosity, optical transparency (for imaging), and be biocompatible and bioactive. The table in FIG. 2 summarizes the advantages and disadvantages of several engineered BMs.

The most widely used in vitro basement membrane model are track-etch polycarbonate (PC), polyester or polyethylene terephthalate (PET), or polytetrafluoroethylene (PTFE). These semi-permeable membranes are commercially available with cylindrical pores of 0.4, 1, 3, 5, or 8 μm-diameter. These membranes are the industry standard for decades and are employed in transwell inserts (manufacturers: Corning, Falcon, etc.), and can also be integrated into microfluidic devices by bonding these membranes between upper and lower microfluidic channels. For barrier models, endothelial or epithelial cells are cultured on one side of the membrane, and unless migration is desired, a small pore size (typically 0.4 or 1 μm) is used to ensure cells remain on a single side of the membrane. These membranes, however, lack physiological relevance in key aspects. First, these membranes are generally 10-12 μm thick (or 30 μm for PTFE), over 100 times thicker than the basement membrane in vivo. This thickness prevents direct cell-cell contact across the membrane and limits the transport of soluble factor transport between adjacent cell types. Second, these membranes have low porosity, in part because the pore spacing is random. Track-etch membranes sold as “high porosity” typically have maximum porosities of around 15% for 0.4 and 1 μm pore sizes and have poor imaging properties. Conventional track-etch membranes have porosities as low as 0.5%. Low porosities decrease the cell-cell crosstalk and reduce diffusion of drugs and molecules across the membrane.

Finally, the flat, sheet-like construction of these membranes cannot mimic the fibrous nature of the basement membrane, are far too stiff for physiological relevance, and are not bioactive. Furthermore, track-etched membranes can also obscure transendothelial electrical resistance (TEER) measurements. TEER is used to measure tight junction integrity (cell-cell adhesion), however cell-substrate adhesion can cause additional electrical resistance that inflates TEER measurements and leads to less consistent results between studies. Finally, track-etched membranes can obstruct brightfield imaging (PC is translucent) and confocal imaging due to autofluorescence. Due to these limitations, new basement models are needed to improve in vitro barrier modeling.

Several basement membranes are under development as alternative to track-etched membranes, such as nanofabricated SiN membranes, electrospun nanofibers, patterned materials such as PDMS and mylar, and native ECM membranes such as collagen and collagen vitrigel membranes. Nanofabricated silicon nitride membranes can be extremely thin (50 nm to 1 μm), have evenly distributed pores, are and optically transparent. However, these membranes require expensive microfabrication processes, are non-fibrous, are not naturally degradable, and can result in cell attachment problems due to the SiN. Anodic aluminum oxides have also been investigated for cell models.

Similarly, photolithographically-patterned Mylar and PDMS membranes have been fabricated with desirable properties such as relatively high porosity (up to about 50%), controlled pore size/location, moderate thickness (generally 5-50 μm, but as low as 2 μm) and are optically transparent. However, these membranes are non-fibrous and require microfabrication. To achieve fibrous membranes, electrospinning techniques have been used. However, these membranes have disadvantages such as large thicknesses (>10 μm), and randomly distributed fibers and pores. Native extracellular matrix (ECM) membranes have also been developed, such as vitrified collagen membranes, but these membranes have low porosity due to the vitrification process and are rather thick (>10 μm). These experimental membranes show improvements to conventual membranes; however, they have yet to gain widespread use. Improved membrane models are needed to reduce the undesirable properties of currently available membranes.

In this disclosure, ultra-thin, ultra-porous fibrous networks fabricated with the Spinneret-based Tunable Engineered Parameters (STEP) technique for basement membrane mimicking networks that offer several advantages compared to other membranes are introduced. First, STEP membranes are ultra-thin, can be fabricated close to physiologically relevant thicknesses. STEP membranes can be fabricated with 1-3 μm thickness, which can be 3×->10× thinner than commercial track-etched membranes. Second, STEP membranes are highly porous. The membrane thickness can be sub-micron or larger. For example, the membrane thickness can be about 5 microns or less, including sub-micron thicknesses. Membrane porosity depends on fiber diameter and spacing, but porosities over 80% can be achieved, which is well beyond the porosity of other reported membrane types (generally <50%). Because the STEP membranes are thin and highly porous, significant crosstalk can be achieved between cells cultured on adjacent sides of the membrane as these cells can make physical contact across the membrane, while still confining cell growth to their respective sides of the membrane.

Third, STEP membranes are fibrous, just like the native basement membrane. Fibrous membranes are more relevant than commercial membranes that are flat plastic sheets with cylindrical pores. Likewise, the fiber networks are more compliant than plastic sheets. Fourth, minimal cell-substrate contact can reduce transendothelial electrical resistance (TEER) inconsistencies. The STEP membranes can be integrated in both microfluidic and transwell experiments. Incorporating STEP membranes into microfluidic devices can enable the study of induced shear stress on individual or both sides of the membrane's co-culture, which is important for tight barriers. Finally, STEP membranes can be made of a variety of polymers (e.g., polystyrene, polyurethane, polylactic glycolic acid (PLGA), collagen, collagen-PLGA blends, hyaluronic acid, polyethylene oxide, etc.), making them highly biocompatible and excellent for cell adhesion. They can be coated with ECM to create a natural surface for cell growth. STEP fibers can also promote easy translation into commercial usefulness.

The results demonstrate that STEP membrane can be used for barrier models and offers significant advantages over existing membranes. The utility of the nanofiber membranes is demonstrated by modelling (mimicking) the blood-brain barrier (BBB) using human endothelial cells and pericytes in monoculture and contact co-culture models. The two cell types have the ability to reach out and make contact. Teer measurements robustly show the marked improvement over standard approaches. The nanofibers can be incorporated into existing Transwell plates or incorporated into microfluidic devices for dynamics experiments. This can demonstrate physiological flow of nutrients across the membranes. Ultra-thin, ultra-porous, fibrous networks fabricated by the STEP technique can provide a more physiologically relevant basement membrane than alternative membrane fabrication methods, leading to improved models of the BBB and other in vitro barrier models for a vast range of applications.

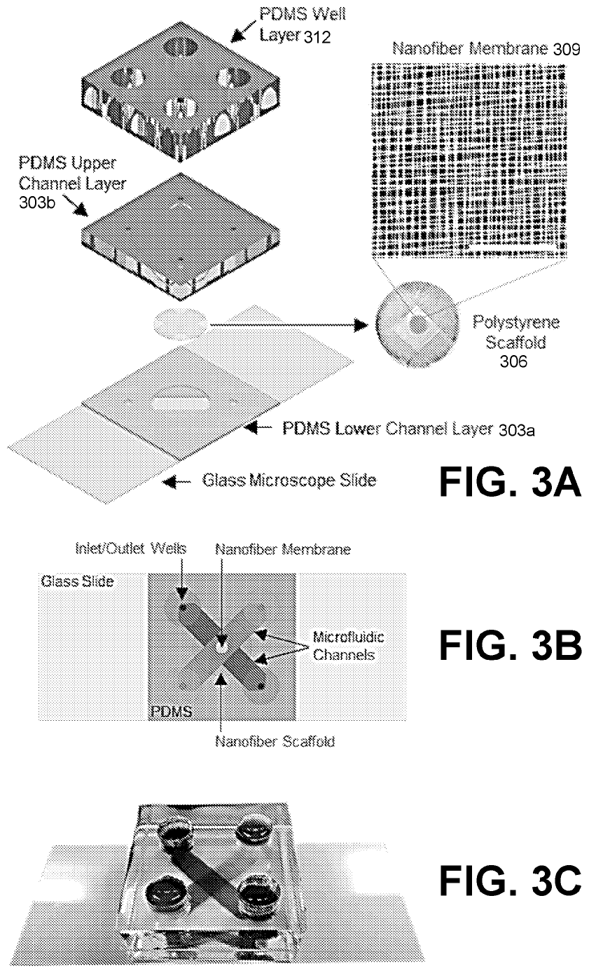

Nanofiber Membranes Embedded within a PDMS Microfluidic Device. Referring to FIGS. 3A-3C, shown is an example of a three-layer PDMS microfluidic BBB chip comprising two orthogonal channels separated by a dense nanofiber network that forms a basement membrane for co-culture BBB models. The channels can be offset by other angles as needed. FIG. 3A provides an exploded view of the microfluidic BBB chip, FIG. 3B provides a top view, and FIG. 3C is an image of a fabricated chip. The scale bars of FIG. 3A is 50 μm. To create the microfluidic model, two pattered polydimethylsiloxane (PDMS) channel layers 303 that enclose a nanofiber scaffold 306 and membrane 309 were fabricated. The channel layers 303 can be formed by other appropriate materials including, but not limited to, elastomers, hydrogels, thermoplastic polymers, of inorganic materials. PDMS layers 303 were formed from laser-cut and solvent bonded acrylic molds. The lower PDMS layer 303a was achieved by pressing two acrylic mold together to pattern both sides of the PDMS. Upper and lower channels were fabricated to be 220 μm tall, 3 mm wide, and 18 mm long. At the intersection of the upper and lower channels, a laser-cut plastic scaffold 306 with nanofiber membrane 309 was embedded, having a nanofiber membrane spanning the 2 mm-diameter circular hollow region. For comparison studies, conventional track-etched PET or PC membranes were bonded to identical laser-cut scaffolds. The scaffolds and membranes were sealed within the device with liquid PDMS glue. Finally, a PDMS well layer 312 was plasma bonded to the top surface of the upper channel layer 303b to enable easy fluid handling.

Ultra-Thin, Ultra-Porous Nanofibers Membranes Fabricated by STEP Method. To replicate the ultra-thin and highly porous in vivo basement membrane, a non-electrospinning Spinneret-based Tunable Engineered Parameters (STEP) method was used to deposit dense networks of nanofibers with highly controlled fiber diameters and fiber spacing. The STEP method uses liquid prepolymer dispensed from a microneedle to deposit nanofibers around a rotating substrate. This method enables a high level of control of fiber diameters (100 nm-10 μm) and fiber spacing (sub-micron and above). The nanofibers are made of polystyrene, and thus highly biocompatible. To create the crosshatch nanofiber pattern, scaffolds are rotated about 90 degrees to deposit orthogonal fibers, and layers can be stacked to create membranes of various thicknesses and pore sizes.

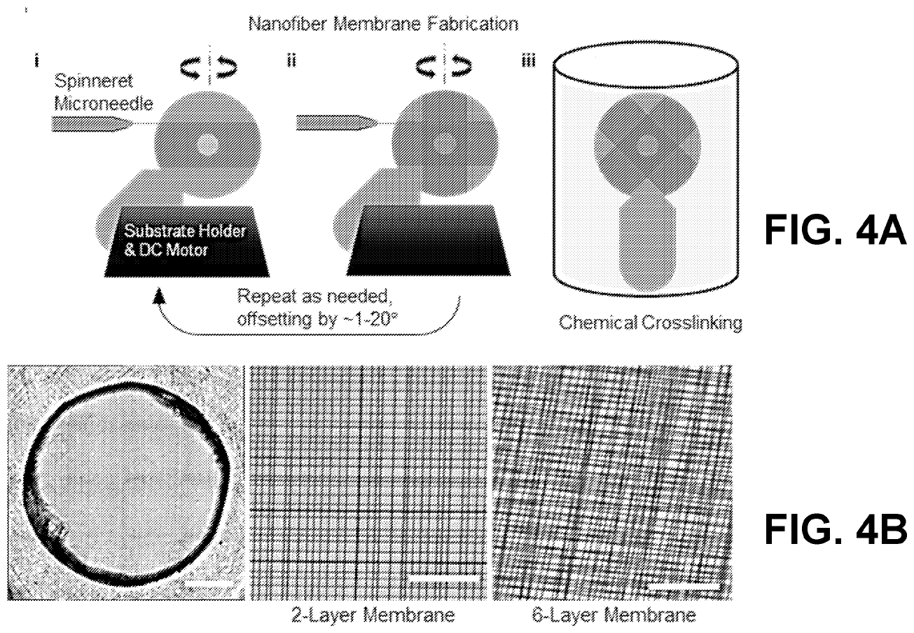

FIG. 4A illustrates an example of the fabrication of polystyrene networks using STEP. Beginning at (i), polystyrene pre-polymer is dispensed from spinneret microneedles and wrapped around a rotating polystyrene scaffold. Next, the scaffold can be re-oriented to spin orthogonal fibers at ii, and the spinning can be repeated to deposit additional fiber layers as desired. The nanofibers are then chemically crosslinked for stability at iii. FIG. 4B shows phase contrast images of examples of nanofiber membranes comprising dense networks of biocompatible polystyrene nanofibers with controlled fiber diameters and fiber spacing. Suspended crosshatch nanofiber patterns were fabricated by stacking orthogonal fiber layers on top of previously deposited fibrous-layers The nanofibers were spun as uniform networks across a 2-mm circular membrane region (left image), and the number of fiber layers regulates pore size. Two-layer (center image) and six-layer (right image) nanofiber networks are pictured. The scale bars are 500 μm, 50 μm, and 50 μm, respectively. The nanofibers are chemically fused at their intersections to improve the stability of the membranes.

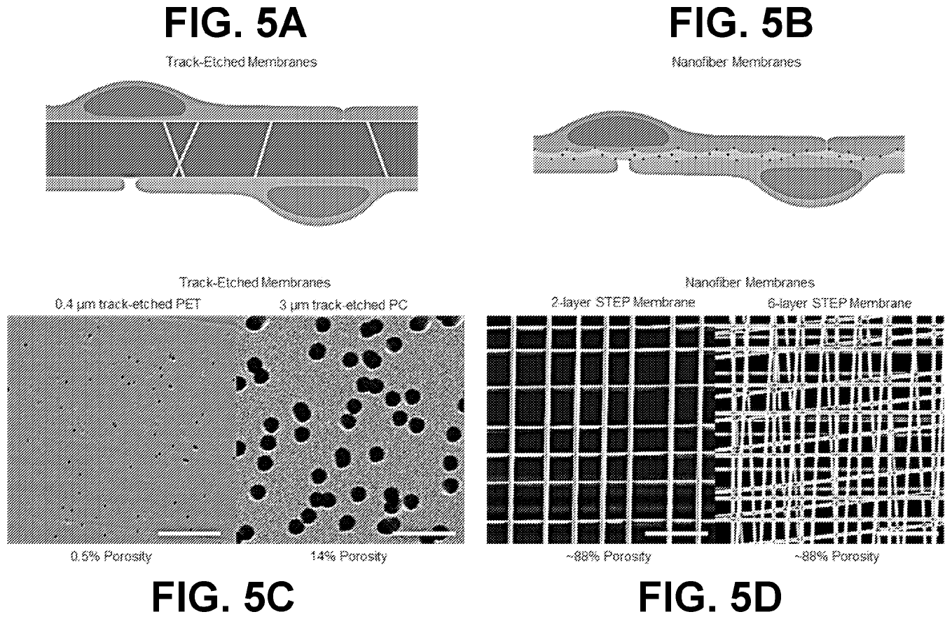

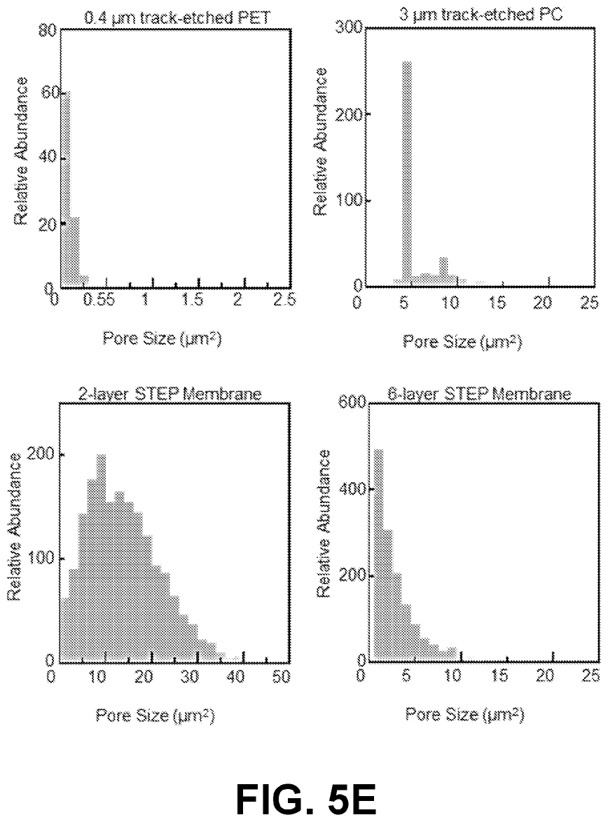

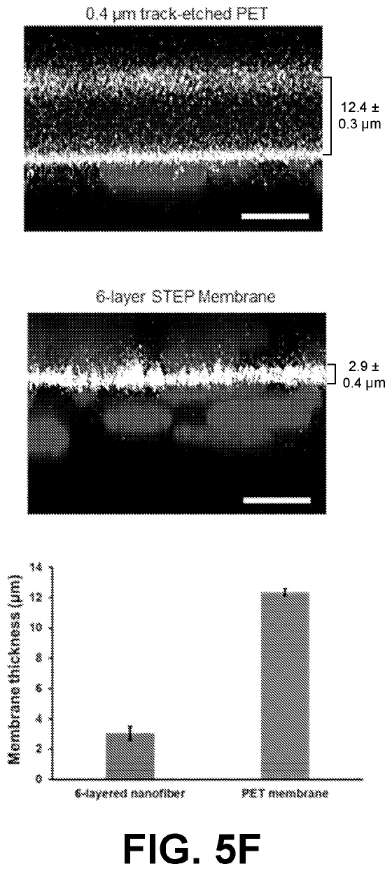

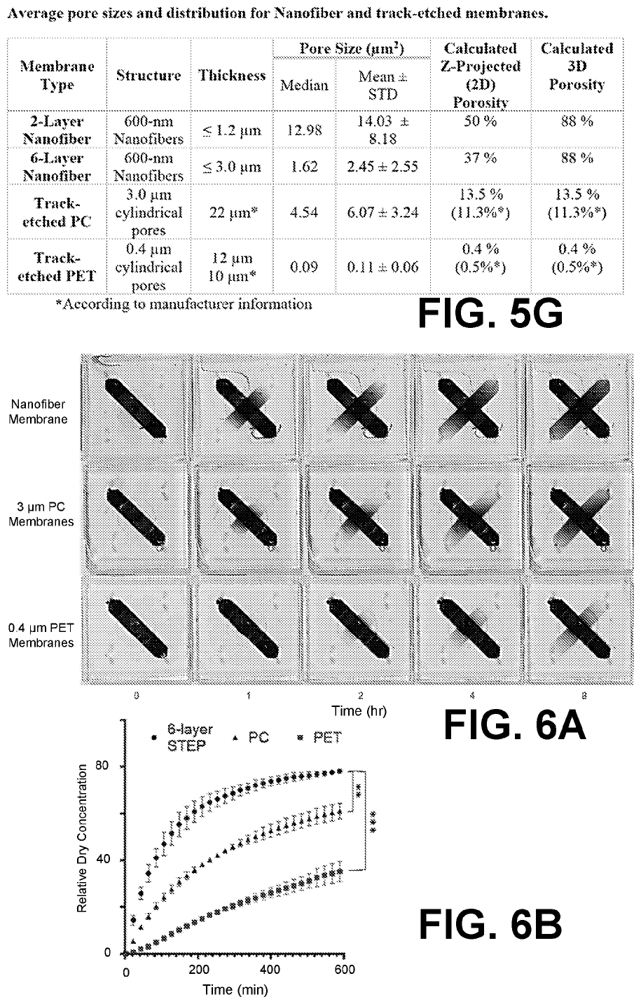

FIGS. 5A and 5B illustrate schematic representations of track etched and nanofiber membranes. Nanofiber membranes are thinner and more porous to better mimic in vivo physiology. FIG. 5C includes SEM images reveal the pore structure of 0.4 μm diameter track-etched PET membranes and 3 μm diameter track-etched PC membranes, and FIG. 5D includes SEM images revealing the structure of 2-layer and 6-layer nanofiber membranes. The 2- and 6-layer membranes were formed with 600-nm diameter fibers spaced approximately 4 μm apart. Track-etched membranes have randomly located pores of uniform size whereas the nanofiber membranes have precisely organized pore structures with a greater distribution of pore sizes. Track-etched membranes have very low porosity of less than 15%. In contrast, the nanofiber networks have porosities of nearly 90%.

Conventional membranes for barrier modeling are 0.4 μm PET membrane, which is shown in the left image of FIG. 5C. Although widely used, these membranes have extremely low porosity of less than 0.5%. Low porosity limits cell-cell interactions across the membrane and creates an unnatural barrier to molecular transport that could decrease the sensitivity of transport studies. Higher porosities can be achieved with polycarbonate (PC) membranes and larger pore sizes (1, 3, 5 μm diameter, etc.), which is shown in the right image of FIG. 5C. For comparison, the porosity of a PC membrane with 3 μm diameter pores was analyzed. These PC membranes still have less than 15% porosity. Furthermore, PC membrane are not optically transparent and thus limit imaging capabilities.

In contrast to conventional track-etched membranes, the STEP nanofiber membranes have extremely high porosities as demonstrated by the SEM images in FIG. 5D. The left and right images show two possible nanofiber membrane configurations: two layers of orthogonal 600-nm diameter fibers spaced approximately 4 μm apart (2-layer membranes) and six layers of stacked 600-nm diameter fibers also spaced 4 μm apart in each layer (6-layer membranes), respectively.

FIG. 5E illustrates the pore size distributions for the four membranes. The nanofiber membranes of FIG. 5D have a wider pore size distribution than the track-etched membranes of FIG. 5C. The 6-layer membranes have an average pore size that is significantly lower than the 3 μm diameter track-etched membrane. The pore size, thickness and porosity of the nanofiber membranes can be controlled by fiber spacing, diameter, and the number of fiber layers. Analysis of the z-projected pore size shows that average pore size for the 2-layer membranes is 14.0±8.2 μm² and the average 6-layer membrane pore size is 2.5±2.5 μm². Given these pore sizes, the 2-layer membranes have roughly equivalent pores sizes as 5 μm diameter track-etched pores (pore size about 19.6 μm²), and the 6-layer membranes have pores sizes roughly between 1 μm and 3 μm diameter track-etched pores (pore sizes 0.8 μm² and 7.1 μm²). The Z-projected (2D) porosity was calculated to be 50% for the 2-layer membranes and 37% for the 6-layer membranes.

Both nanofiber membranes considerably outperform the standard track-etched membranes. However, true porosity is a 3D property (volume of empty space to total volume). Assuming membranes have nominal thicknesses based on the fiber diameters (fiber thickness are additive—minimal flattening during chemical fusion), membrane porosity is independent of the number of stacked nanofiber layers, but is rather dependent only on average fiber diameter and average fiber spacing. For both the 2-layer and 6-layer scaffolds, which have 600 nm fibers spaced 4 μm apart, the theoretical porosity for both membranes are 88%. This represents a 220× increase over the 0.4 μm pore PET membranes and a 6.5× increase over the 3 μm PC membranes. At almost 90% porosity, these membranes are near the limit of what might be experimentally possible, and higher porosity membranes are not likely to significantly increase transport across the membranes. High porosity membranes have the potential to increase the crosstalk between adjacent cell types to physiologically relevant levels.

In addition to the ultra-high porosities of the nanofiber membranes, the membranes are also significantly thinner than conventional track-etched membranes. Conventional track-etched PET and PC membranes range from 10 to 12 μm in thickness as shown in the left image of FIG. 5F, over 100× the thickness of the basement membrane in vivo. The nanofiber networks offer at least a 3-fold decrease in thickness. The 2-layer membranes are nominally 1.2 μm thick, while the 6-layer membranes are nominally 3 μm thick as shown in the right image of FIG. 5F. Rhodamine fibronectin (white) indicates membrane surfaces (scale bar 10 μm). The bar chart of FIG. 5F graphically illustrates the difference in thicknesses between the membranes. The chemical-fusion process can cause the nanofiber intersection points to melt and decrease the overall membrane thickness below nominal levels, but at the cost of also reducing the porosity of the membrane. At <3 μm thick and highly porous, the membranes can enable significant cell-cell interactions across the membrane. The pore size and distribution information are summarized in the table of FIG. 5G.

Nanofiber membranes enhance trans-membrane molecular diffusion. The combination of high porosity and low thickness of the nanofiber membranes enable significantly enhanced the molecular diffusion across the membrane. To visually demonstrate increase trans-membrane diffusion, time-lapse diffusion experiments were performed using food dye. FIG. 6A illustrates the diffusion of food dye from the lower channel to the upper channel (from left to right). As can be seen, the 6-layer nanofiber membranes (top row) enables significantly enhanced diffusion (increased rate of dye diffusion) from the lower channel to the upper channel compared with PC (center row) and PET (bottom row) membranes. The increase is especially pronounced between the 0.4 μm PET membrane and nanofiber membrane. During experiments, inlet and outlets were blocked to eliminate fluid flow. The amount of dye concentration was quantified by color intensity and normalized relative to the bottom channel. The results are shown in FIG. 6B.

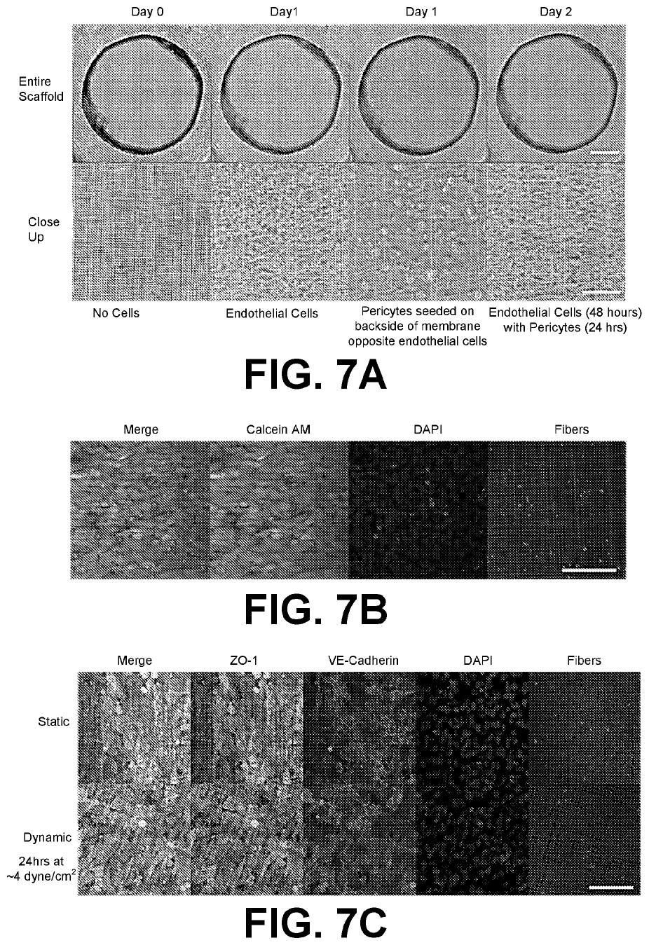

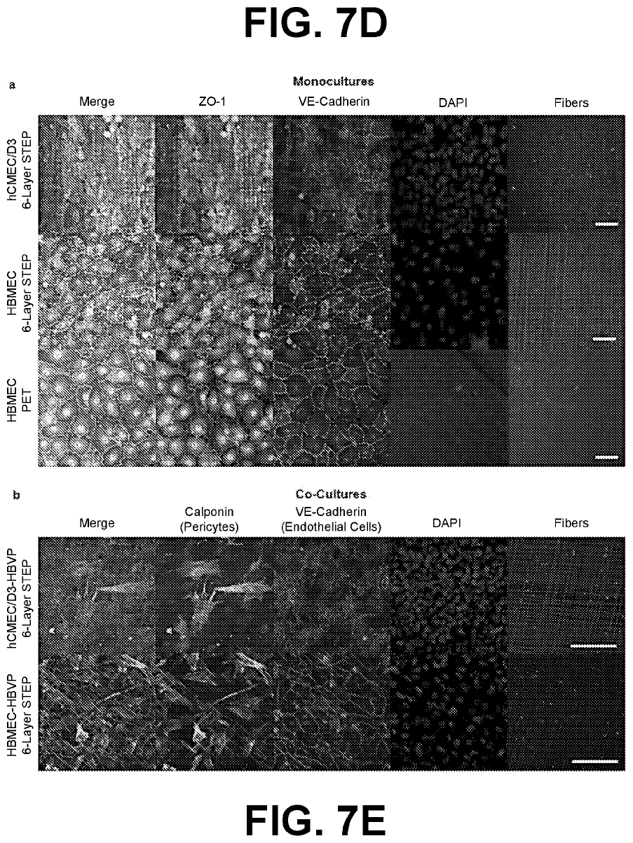

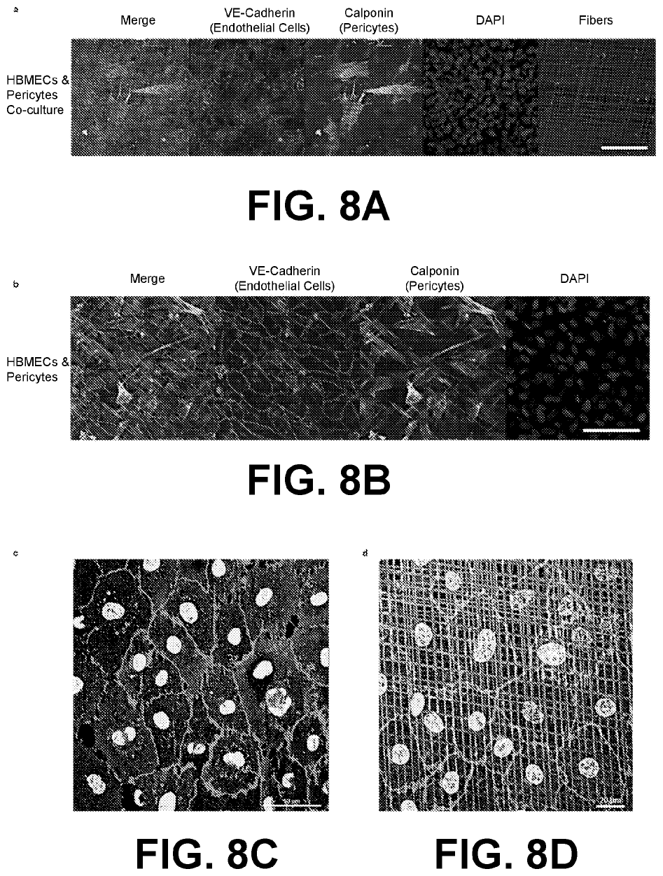

Nanofiber membranes support endothelial monolayer formation. Given the superior properties of the nanofiber membranes compared to track-etched alternatives, whether endothelial monolayers could be formed on the fiber membranes, and thus could be used to model physiologically relevant basement membrane biology in vivo, was investigated. It was found that two human brain microvascular cell lines were able to form intact monolayers: immortalized human cerebral microvascular endothelial cells (hCMEC/D3) and primary human brain microvascular endothelial cells (HBMECs). Endothelial cells were seeded at high densities on the fibers, and monolayers formed within 24 hours. FIG. 7A shows brain endothelial cells (hCMECs) seeded on 6-layer membranes on day 0 form confluent monolayers after 24 hours (day 1). At 24 hours (day 1), pericytes can be added to the opposite side of the membrane. By day 2, pericytes have spread on the fibers to form a stable co-culture BBB model.

FIG. 7B shows a complete monolayer of hCMECs formed after 72 hours of culture on the fibers. Calcein-AM staining on live endothelial cells (hCMECs) after 72 hours of monoculture show complete monolayer formation on the nanofibers. Furthermore, confocal microscopy reveals that endothelial cells are confined to one side of the membrane. Nanofiber are stained with rhodamine-conjugated fibronectin. FIG. 7C shows the morphology after 48 hours of culture in static and dynamic (oscillation flow for 24 hours, max shear stress about 4 dynes/cm²). Antibody staining of endothelial cells (hCMECs) for the tight-junction protein ZO-1 and the endothelial adhesion molecule VE-cadherin show localization at cell boundaries indicating barrier formation after static or dynamic culture. Both monolayers show enhanced expression of ZO-1 (tight junctions) at cell boundaries, and significant VE-cadherin expression at cell boundaries.

To explore the integrity of the monolayers, the expression of tight junction markers ZO-1 and VE-cadherin for both monolayer cultures of HBMECs and hCMECS/D3s was interrogated. FIG. 7D illustrates examples of immortalized endothelial cells (hCMEC/D3s, top row) and primary human brain vascular endothelial cells (HBMECs) cultured in monolayer on nanofibers (middle row) and PET membranes (bottom row). Expression of ZO-1, VE-Cadherin, DAPI, and rhodamine-conjugated fibronectin are shown. Scale bars 20 μm. FIG. 7E illustrates that both endothelial cell lines had significant expression of ZO-1 and localization of VE-Cadherin to cell borders. HBMECs had almost ubiquitous localization of expression at the borders between cells, showing both excellent tight junction expression and monolayer formation. The STEP membrane and PET membrane have comparable expression, but the nanofiber membrane appears noisier than the PET membrane. This may be attributed to the fibrous 3D topography of the nanofiber membrane, compared to the flat 2D surface of the PET membrane. 7E illustrates examples of hCMEC/D3s (top row) strained with VE-Cadherin and primary pericytes (HBVPs) stained for calponin demonstrate successful co-culture across the nanofiber membrane. HBMECs (bottom row) likewise show characteristics of monolayer formation along with pericyte localization. Scale bars 20 μm.

HBMEC cells showed significant localization of VE-Cadherin to cell borders. Co-cultures of endothelial cells with pericytes enable cell-cell interactions across the nanofiber membranes. FIG. 8A illustrates examples of human endothelial cells (hCMECs) stained with VE-Cadherin and primary pericytes stained for calponin demonstrate successful co-culture across the nanofiber membranes. FIG. 8B illustrates examples of primary human endothelial cells (HBMECs), which likewise show characteristic monolayer formation along with pericyte localization. FIGS. 8C and 8D show higher magnification images showing VE-cadherin localization to cell boundaries, and monolayer formation on the nanofiber membrane (with rhodamine fibronectin shown), respectively. Scale bars 20 μm.

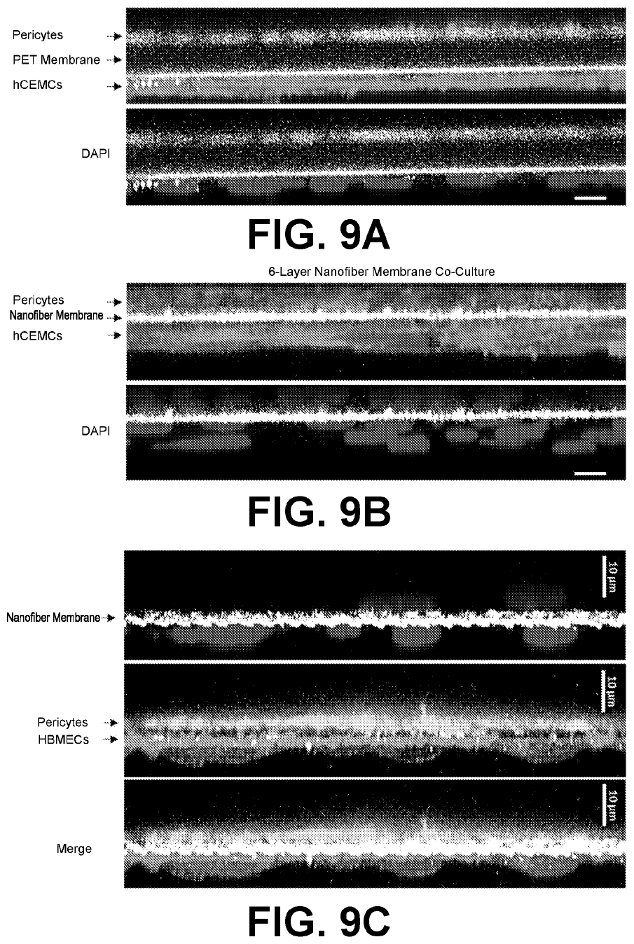

Nanofiber membranes enable cell-cell contact co-cultures for BBB studies. Due to the low thickness and high porosity of the membranes, culturing pericytes and endothelial cells on opposite sides of the nanofiber membrane was sought to mimic the spatial configuration of these cells in vivo. To perform these experiments, endothelial cells were first seeded onto the underside of the nanofiber membrane and allowed to spread and form a monolayer for 24 hours. Pericytes were then added on the topside of the membrane and allowed to spread for 24 hours, after which devices were fixed and stained. FIG. 7A shows phase contrast images over the course of an experiment. As shown in FIGS. 9A-9C, pericytes were successfully cultured opposite endothelial monolayers for both hCMEC and HBMEC cells.

Z-stack images of co-cultures demonstrate the close-contact of pericytes and endothelial cells across the nanofiber membranes. Actin is shaded, and rhodamine-fibronectin on the membrane is pictured was white. FIG. 9A illustrates an example of PET track-etched membranes show significant separation between pericytes (top) and endothelial cells (bottom) which prevents cell-cell interaction. In contrast, 6-layer nanofiber membranes shown in FIG. 9B are significantly thinner and enable cell-cell interactions across the membrane. Notice that the imaging quality on upper side of the PET membrane is significantly reduced due to the mismatch in refractive index of the plastic. There is minimal resolution loss across the nanofiber membranes. FIG. 9C shows Z-stack images illustrating the narrow separation of primary endothelial cells (VE-Cadherin) and pericytes (calponin) in co-culture models. As shown, HBMEC cells and pericytes are in very close proximity during co-culture studies.

It can be seen from confocal images that the nanofiber membranes are significantly thin compared to the tracked-etched membrane. Rhodamine fibronectin (white) was used to promote cell adhesion and visualize the membranes, and cell nuclei are stained with DAPI. For the track-etched membrane, there are two distinct lines of expression on the top and bottom of the membrane, with the bottom being noticeably more visible. The membranes are not optically transparent and impede imaging through them; furthermore, they are auto-fluorescent, emitting blue light when excited. This may make it difficult to visualize entities that share similar wavelengths. The nanofiber membrane, however, does not have either disadvantage. In contrast, visible separation of rhodamine expression across the 6-layer STEP membrane was not observed, and minimal loss of image resolution through the STEP membranes was found, indicated by the increase in resolution for cells on top of the membrane.

Unlike conventional tracked-etched membranes which are flat, the fibrous nature of the nanofiber membrane allows for a 3D environment for the cells to attach to. The cells do not form focal adhesion on a 2D surface but can reach beyond the top and bottom nanofiber layer into the open space between nanofibers. This may allow cells in co-culture to reach into the space and come into contact with each other.

Results. It has been shown that precisely aligned nanofiber networks can be used to create physiologically relevant basement-membrane mimics. Current basement membrane models lack physiological relevance, such as unrealistic thicknesses (generally >100 times greater than in vivo thickness), low porosity (generally <15%, but often as low as 0.5%), and sheet-like (flat) rather than fibrous construction. Thus, currently available semi-permeable membranes used for barrier modeling lack important properties that would enable more accurate barrier models, especially cell-cell contact co-culture models. To overcome limitations of conventional barriers, an advanced yet simple non-electrospinning technique known as STEP was used to fabricate dense networks of highly ordered polystyrene nanofibers.

Nanofiber network properties, such as fiber diameter spacing, fiber diameter, fiber orientation, and number of fiber layers can be precisely controlled to create membranes with variably thickness and porosity. Dense networks polystyrene nanofibers can be used to create ultra-thin (about 1-3 μm), ultra-porous (nearly 90% porosity), fibrous basement membranes for co-culture barrier models. The nanofiber membranes can significantly exceed the highest reported porosities for membranes used in barrier modelling. While the described 2-layer and 6-layer nanofiber membranes form basement membranes with superior properties, these membrane properties may be optimized using different fiber diameters, especially smaller diameter fibers. Nanofibers as small as 100-nm in diameter can easily be fabricated with the STEP method. Incorporating smaller diameter fibers can produce membranes that nearly reach in vivo membrane thicknesses. Due to the fragile nature of 100-nm diameter fibers, 600-nm diameter fibers were chosen for this study due to their added strength. Using large diameter fibers as structural supports for smaller diameter fiber networks may enable strong yet extremely thin basement membrane mimics.

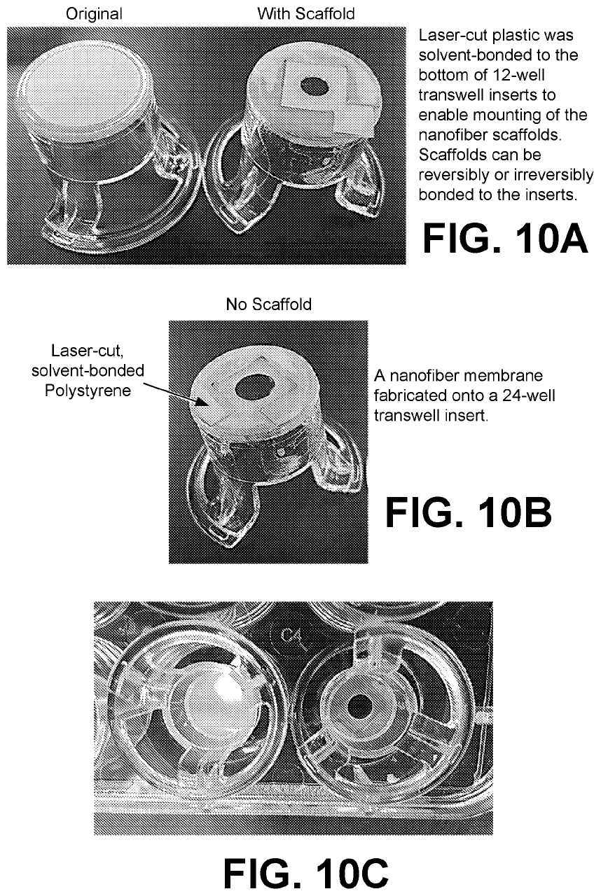

The nanofiber membrane was integrated into a microfluidic barrier model. Microfluidic models have become increasing prominent in barrier research, as these devices enable cell confinement, complex geometries, and fluid shear stress. The device was fabricated from PDMS. However, absorption and adsorption of molecules by PDMS makes it unfavorable for drug transport studies. Since the nanofibers utilize polystyrene, an entirely plastic device can be designed and assembled for commercial use. Furthermore, the nanofibers can be integrated into conventional transwell plates, enabling use with current well plate-based equipment and rapid adoption. As a proof-of-concept, a conventional 24-transwell plastic insert was modified to accept the nanofiber scaffold. FIGS. 10A-10C include images illustrating an example of a transwell plate (or insert) modified to accept a nanofiber scaffold. FIG. 10A shows an original transwell insert (left image) and the modified transwell insert comprising the nanofiber scaffold with a nanofiber membrane (right image). The nanofiber membrane is positioned over an opening formed in the transwell insert. FIG. 10C shows the opening formed in the bottom of the transwell insert. A laser-cut polystyrene guide was solvent-bonded to the bottom surface of the transwell insert. The nanofiber scaffold is aligned with the guide to place the nanofiber membrane across the opening and bonded to the bottom surface of the transwell insert. FIG. 10C shows the original and modified transwell inserts positioned within a tray.

Due to the advantages of microfluidics, the experiments were performed in the microfluidic device. The ability to add fluid shear stress, which is known to be an important mechanical stimulus for improving barrier tightness via cytoskeletal changes and tight-junction formation, provides an advantage. It was found that static culture conditions produced good barriers, and the addition of oscillating shear flow was investigated. Although 3D hydrogel models of the BBB show significant potential, the added complexity of these devices has thus far limited their commercialization. Due to the incorporation of gel the device, measurement of absolute barrier permeability (TEER or molecular diffusion) is generally not possible or very difficult. Furthermore, these models are not able to collect the effluent from the models to measure the concentration of a molecule or drug that was transported across the membrane. Consequently, 2D shear models with engineered membranes will continue to be used to research efforts for BBB modeling and other barrier modelling.

Endothelial monolayers were cultured on the nanofiber scaffolds and demonstrated that the monolayers express junctional proteins such as ZO-1 and VE-Cadherin. Confocal imaging shows that endothelial cells were constrained to only one side of the nanofiber membrane. Furthermore, pericytes were co-cultured on the opposite side of the membrane and the highly porous and thin membrane was shown to enable close contact between endothelial cells and pericytes.

Using integrated electrodes, TEER values were measured across the nanofiber scaffold, and it was found that TEER values increased after seeding endothelial cells across the membrane. TEER values for the BBB in vivo have been measured to be >1000 Ω/cm². Most in vitro models fall significantly below this value. The TEER measurements are considerably lower compared with other reports, however this may be attributed to the geometry of the device and the small size of the nanofiber membrane. Importantly, both PET and the nanofiber membranes showed similar TEER values during the first three days of cell culture. The BBB model may be further improved by including human-induced pluripotent stem cells (hIPSC) that have been shown to have higher TEER values and would enable patient-specific models. Likewise, alternative attachment factors such as Matrigel could be explored to further increase barrier tightness.

TEER values taken across nanofiber membranes can yield more consistent results compared to track-etched membranes. The inconsistency in TEER values reported in literature may be attributed to differences in track-etched membranes and not actually reflect the tightness of endothelial junctions. For example, track-etched pore diameter and density significantly influence the electric field current paths. Furthermore, due to the flat sheet-like nature of track-etched membranes, cell-substrate adhesion can influence TEER values by artificially inflating values. These sources of error are consistent with reports that surface roughness of the track-etched membranes and pore size influence TEER values. The highly porous nanofibers may eliminate most of the error from both cell-substrate adhesion and from distortions in the current paths due to the highly porous nature of the membranes.

The nanofiber networks have significant advantages for studying cell and nanoparticle migration across the BBB due to their controllable pore size and high porosity. Conventional track-etched membranes are unsuitable for nanoparticle transport studies due to the adhesion of nanofibers to the membranes and within track-etched pores. The highly porous membrane can minimize interference of the membrane to nanofiber transport. Likewise, modelling cancer metastasis and immune cell migration across the BBB is of significant important for researchers, and the models can enable high-levels of migration due to the high porosity and low membrane thickness. The larger pore sizes (generally ≥3 μm pores) used for cellular transmigration studies have the disadvantage of allow endothelial cells to cross the membrane and for a second monolayer on the opposite side of the membrane. The thin, highly porous membranes can offer advantages for such studies.

The majority of pericytes are confined to one side of the membrane, however, in some instances it appears that pericytes can migrate across the membrane pores and interact with the endothelial monolayer more closely. Previous studies have shown that migratory astrocytes that cross track-etched membranes for pores as small as 3 μm in diameter. Migratory pericytes or astrocytes across the membrane clearly is not consistent with the in vivo configuration, but is more realistic for the thin membranes.

In the experiments, the nanofiber membrane area was limited to a circle of 2 mm diameter (0.031 cm²). A small membrane area was chosen to reduce manufacturing time of each membrane and constrain the microfluidic chip to a small size. However, the membrane size is small compared with transwell plates (0.143 cm² for some commercial 96 transwell designs). The size of the nanofiber scaffolds may be increased up to about 3 cm² (1-cm circle). Larger membrane areas, however, have longer fabrication times and are less strong. Large nanofiber membrane may need additional support to prevent the delicate fibers from being disrupted by liquid handling and other experimental procedures.

One consideration when using these nanofiber membranes is that, due to the highly porous nature of these membranes, even slight pressure gradients across the network can cause significant trans-membrane fluid flow. The highly porous nanofiber membranes act more like open channels than walls/barriers. This characteristic makes fluid handling more challenging with nanofiber membranes compared with track-etched membranes. For instance, transwells are generally inverted while seeding endothelial cells on the bottom of the membrane. A similar method for seeding cells on the bottom of a nanofiber membrane would not work, as the liquid would pass through the membrane rather than forming a droplet on top. This channel makes microfluidics a natural choice since fluid handling is performed within the chip and the entire device can be inverted.

Using an advanced, yet straightforward fabrication technique, ultra-thin, ultra-porous networks of polystyrene nanofibers have been generated that function as in vitro basement membranes and significantly increase the physiological relevance of commonly used semipermeable membranes. A microfluidic model of the blood-brain barrier was constructed using human endothelial cells and pericytes, demonstrating monolayer formation and cell-cell contact across the nanofiber membrane. The nanofiber membranes can improve the realism of physiological barriers for in vitro endothelial and epithelial barrier models and offer new opportunities to modeling human physiology, drug discovery, cell migration studies, and barrier dysfunction studies.

### Methods

Nanofiber fabrication. Dense nanofiber membranes were fabricated according to the previously published Spinneret-based Tunable Engineered Parameters (STEP) method, schematically illustrated by FIGS. 4A and 4B. Further details regarding the STEP method are provided in U.S. Pat. Nos. 9,029,149, 9,753,023, and 9,902,932, which are hereby incorporated by reference in their entireties. Nanofibers were spun around scaffold supports that were laser-cut from plastic coverslips (e.g., Fisher Scientific, 0.22 mm thick) and contained a hollow region 2 mm in diameter over which the nanofiber membrane was fabricated. Liquid prepolymer was dispensed from a microneedle and nanofibers were spun around a rotating and translating scaffold substrate. The scaffold was then re-oriented and spun again to create orthogonal fiber. The 600 nm diameter fibers were prepared using a 7 wt % solution of polystyrene (e.g., MW: 2000 000 g/mol; category no. 829; Scientific Polymer Products, Ontario, NY, USA) in p-xylene (e.g., X5-500; Thermo Fisher Scientific, Waltham, MA, USA). To decrease pore sizes, multiple nanofiber layers were spun across the scaffold. Nanofibers were then chemically fused together. To improve the throughput of the spinning process, and to eliminate the need to remove unwanted fibers from one side of the scaffold, two scaffolds were secured back-to-back by epoxying their stubs together. Prior to integrating them in the devices, the scaffold stub was carefully cutoff.

Microfluidic fabrication. Microfluidic device was fabricated from two polydimethylsiloxane (PDMS) layers that enclosed a plastic nanofiber scaffold. PDMS was mixed 1:10 base to cross-linker and was cured in acrylic molds at 50 C for 4 hours. Acrylic molds assembled from laser cut and solvent bonded layers of acrylic. The lower microfluidic channel and the indentation for the nanofiber scaffold were patterned using a top and bottom acrylic mold pressed together by binder clips. Access holes for the inlet and outlets of the lower channel were punched in the lower PDMS membrane using a 1.5 mm-diameter biopsy punch, and the PDMS layer was plasma bonded (Harrick Plasma) to a glass slide. The upper microfluidic channel was similarly formed in an acrylic mold, and once cured inlet and outlet ports were punched in the upper layer using a 1 mm-diameter biopsy punch. Finally, a top well layer was cured in an acrylic mold and 6 mm diameter holes were punched with a biopsy punch. Upper and lower microfluidic channels were 0.22 mm high, 3 mm wide, and 18 mm long. Liquid PDMS glue was used to bond the nanofiber scaffold between the upper and lower channel layers. Liquid PDMS was spun at 3000 RPM for 1 minute on a glass slide and de-gassed upper PDMS layers were placed channel-side down on the PDMS layer to absorb the PDMS. Liquid PDMS was loaded into a 1 ml syringe connected to a dispensing needle. A small amount was spread on the lower PDMS layer where the indentation for the scaffold was located. The nanofiber scaffold stub was then carefully cut-off, and the scaffold was placed fiber-side down on the lower PDMS layer. The plastic scaffold containing the nanofiber network (or PET membrane for comparison) was then placed in the circular indentation and adhered in place with liquid PDMS glue.

Track-etched membranes. To enable a comparison between the nanofiber membranes, track-etched polyethylene terephthalate (PET) or polycarbonate (PC) membranes were also incorporated into the microfluidic device. PET membranes were removed from 6-transwell plates (e.g., Corning, #3450) and according to the manufacturer were 10 μm thick, had 0.4 μm diameter pores at 4×10⁶ pores/cm². PC membranes (e.g., Millipore Sigma, TSTP02500) were also tested and according to the manufacturer were 22 μm thick, had 3.0 μm pores with a porosity of 11.3%. Both PET and PC membranes were carefully cut into about 4×4 mm squares and bonded to the laser-cut nanofiber scaffolds using liquid PDMS glue. Liquid PDMS was spun at 3000 RPM on a glass slide. One side of the plastic scaffold was placed in contact with the glue, and then removed. The PET or PC membrane was carefully placed across the 2 mm hollow region, and then cured at 55° C. for 2 hours.

Cell Culture. Human endothelial cells and pericytes were used to model the BBB. Cell culture was performed according to supplier protocols. Human cerebral microvascular endothelial cells (hCMEC/D3, EMD Millipore) were maintained in EngoGrow™-MV Complete media supplemented with 1 ng/mL FGF-2 (e.g., MilliporeSigma), and 1% penicillin/streptomycin (e.g., Life Sciences). Primary human brain microvascular endothelial cells (HBMECs, Cell Systems) were cultured in Cell Systems Medium supplemented with 5 mL CultureBoost™. Primary human brain vascular pericytes (HBVP, ScienCell) were cultured in pericyte basal medium supplemented with 10 ml of fetal bovine serum, 5 ml of pericyte growth supplement, and 5 ml of penicillin/streptomycin solution. Cells were incubated at 37° C. and 5% CO₂.

Fluorescence microscopy and cell staining. Antibody staining was performed to investigate cell morphology. Cells were fixed and stained within the microfluidic devices. Cells were fixed in 4% paraformaldehyde for 15 minutes, washed with PBS, and permeabilized in 0.1% Triton-X-100 in PBS for 15 minutes. Cells were then washed with PBS and blocked with 5% normal goat serum in PBS for 15 minutes. Cells were incubated with primary antibodies mixed in antibody dilution buffer consisting of PBS supplemented with 1:100 w/v Bovine serum albumen (e.g., BSA) and 1:333 v/v Triton X-100. Endothelial cells were stained for ZO-1 (ZO-1 Polyclonal Antibody, Invitrogen #40-2200, 1:100), VE-Cadherin (CD144 (VE-cadherin) Monoclonal Antibody (16B1), eBioscience, #14-1449-82, 1:100), and/or CD31 (Human CD31/PECAM-1 Antibody, R&D Systems, #BBA7, 1:33). Pericytes were stained for calponin (e.g., Abcam, Recombinant Anti-Calponin 1 antibody [EP798Y] (ab46794), 1:200). Cells were incubated with primary antibodies overnight at 4 C. Cells were washed with PBS, and secondary antibodies (e.g., Alexa Flour 488, 555, or 647 at 1:400 in antibody dilution buffer) were added to the device for 45 minutes in the dark at room temperature. Finally, cells were washed with PBS, incubated with 300 nM DAPI in PBS for 10 minutes, and then flushed with PBS for imaging.

For live cell imaging, cells with Cell Tracker Green/Deep Red, Calcein-AM, and/or Nucblue. Calcein-AM staining was performed with 4 μl/ml Calcein-AM in cell culture media and incubated for 15 minutes. Two drops of Nucblue were added to culture media and incubated for 15 minutes. For Cell Tracker experiments, endothelial cells were seeded within the device and allowed to attached and form a monolayer for 24 hours. Endothelial cells were then incubated for 45 minutes in 5 μg/ml CellTracker™ Green CMFDA (Invitrogen, #C2925) in serum-free media within the device. The device was then flushed with media. Pericytes were then passaged and suspended in 1 μg/ml CellTracker™ Deep Red dye (Invitrogen, #C34565) for 45 minutes in serum-free media. The pericytes were then centrifuged twice (1000 RPM for 5 minutes) and added to the microfluidic device. One day after staining the pericytes and endothelial cells, the cells were fixed in place with 4% paraformaldehyde and imaged on a confocal microscope.

Nanofibers were stained with rhodamine-conjugated fibronectin (Cytoskeleton Inc.) at 4 μg/ml overnight.

Diffusion Experiments. Diffusion experiments were performed by adding McCormick Culinary Blue Food Color Dye (Mixture of Blue 1 (MW: 792.85 Da) and Red 40 (496.42 Da)). To prevent dye flow across the membrane, packing tape was used to seal the inlet and outlet ports of the upper channel while filling the device. To decrease evaporation from the device, the entire device top of the device was then sealed with packing tape. Images were collected every minute for 10 hours at room temperature. Diffusion data was than analyzed in ImageJ to determine the time constants of diffusion.

Experimental methods. To prepare the microfluidic devices for experiments, vacuum-degassed devices were sterilized with ethanol and then washed with PBS. The devices were incubated overnight at 37° C. with 4 μg/ml of human fibronectin in PBS. For imaging experiments, rhodamine-conjugated fibronectin (Cytoskeleton Inc) was used at the same concentration. The following day, devices were flushed with endothelial media and incubated for another hour at 37° C. Endothelial cells were passaged according to supplier instruction and suspended at 1×10⁷ cells/ml (2,200 cells/mm²) for the hCMEC cells or at 5×10⁶ cells/ml (1,100 cells/mm²) for the HBMECs cells in cell culture media. Before pipetting the cells into the lower channel of the device, the inlet and outlet of the upper channel were blocked with PDMS-clogged pipet tips to prevent fluid flow across the membrane.

Cells were then added to the lower channel and the devices were immediately inverted and incubated at 37° C. for three-four hours. The devices were then place right-side up, the pipet tips removed, and additional media was added to the device wells. Devices were incubated overnight to allow endothelial monolayer formation. The following day, pericytes were passaged according to supplier instructions and resuspended in pericyte media at 5×10⁶ cells/ml (1,100 cells/mm²). PDMS-clogged pipet tips were used to block the lower channel inlet and outlets, and the cell suspension was added to the upper channel. After incubation for three-four hours, the pipet tips were removed, and additional media was added. Pericytes were allowed to spread and interact with the endothelial cells for 24 hours, after which time the device were fixed with 4% paraformaldehyde for imaging.

For TEER devices having an increased channel height, the cell suspension densities were adjusted to maintain 2,200 cells/mm² or 1,100 cells/mm² for the hCMEC and HBMECs/pericytes respectively. For consistent TEER measurements, TEER devices were initially incubated with a 50:50 mix of endothelial and pericyte media, and all cells were suspended in 50:50 media before adding to the device.

Transwell Experiments. To explore tight junction integrity for both mono- and co-culture models, the custom scaffolds were incorporated into a 24 well transwell system. The same scaffolds and membranes for the microfluidic devices were used, as the scaffolds is the same diameter as the bottom of a 24 well plate transwell insert (e.g., CellTreat, #230635). The current membranes on the bottom of the transwell inserts were removed and liquid PDMS was used to seal the custom scaffolds in place, with the membrane facing the bottom of the insert. The transwells were sterilized with ethanol and UV light. 50 μg/ml of human fibronectin was added to the top of an inverted transwell and incubated for 2 hours at 37° C. The transwells were then inverted into the 24 well plate and media was added.

Initial TEER values were taken to measure the membrane resistances. The transwells were then removed and re-inverted. Endothelial cells and pericytes were passaged and seeded at the same concentration as with the microfluidic devices. The endothelial cells were added and allowed to attach for two hours at 37° C. The transwell were then again inverted into the 24 well plate and media was added. HBMEC attachment was verified with a light microscope. The transwell devices were allowed to incubate overnight. HBVPs were added to the inside of the transwell the following day. TEER measurements for day 1 were taken prior to adding HBVPs. TEER measurements were then taken every day for 5 days, directly after removing from the incubator to maintain the temperature at 37° C. Media was replace everyday post measurements. Barrier formation was verified on day 2 with 4 μM Calcein-AM live-dead stain added directly to the transwell.

Porosity and pore size calculations. Pore size distributions for the nanofiber membranes and track-etched membranes were determined by thresholding Scanning Electron Microscope (SEM) images of the membranes using ImageJ's particle analysis menu. Z-projected (2D) porosity values were also calculated from SEM image in ImageJ by comparing the sum of pore areas with the total imaging area. Actual, 3D porosity values can be estimated based on fiber diameters and fiber spacing. Porosity can be calculated as:

$$Porosity = \frac{{V}_{M} - {V}_{F}}{{V}_{M}} ,$$

where V_{M} is the total volume of the membrane and V_{F} is the total volume of the fibers. Since both V_{M} and V_{F} depend on linearly on the number of layers, the number of layers does not affect porosity values. Rather, porosity is determined by the fiber diameter and fiber spacing. Porosity of the 2-layer and 6-layer membranes were estimated using nominal 600-nm diameter fibers and 4 μm spacing.

Impedance Spectroscopy and TEER measurements. Transendothelial electrical resistance measurements (TEER) were performed using a potentiostat (Gamry, Reference 600). Electrical impedance spectroscopy was performed from 10 Hz to 1 MHz with 10 measurements taken per decade. The applied potential was 0.1 V. A four-electrode configuration was used as described. Impedance measurements were performed at 37 C every 24 hours. TEER values were determined by fitting a circuit model to the impedance spectrum and finding the best-fit values.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

The term “substantially” is meant to permit deviations from the descriptive term that don't negatively impact the intended purpose. Descriptive terms are implicitly understood to be modified by the word substantially, even if the term is not explicitly modified by the word substantially.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include traditional rounding according to significant figures of numerical values. In addition, the phrase “about ‘x’ to ‘y’” includes “about ‘x’ to about ‘y’”.

1. A microfluidic chip, comprising:
a first channel layer comprising a first fluid channel;
a second channel layer comprising a second fluid channel configured to cross the first fluid channel; and
a scaffold disposed between the first and second channel layers, the scaffold comprising a nanofiber membrane separating the first and second fluid channels, the nanofiber membrane comprising a stack of nanofiber layers, each nanofiber layer comprising nanofibers disposed with a controlled orientation and with a controlled spacing, the nanofibers of each nanofiber layer cross-linked with nanofibers of an adjacent nanofiber layer and oriented at an angle with respect to the nanofibers of the adjacent nanofiber layer, the angle in a range between zero and 90 degrees.

2. (canceled)

3. The microfluidic chip of claim 1, wherein the nanofiber membrane has a thickness from about 5 μm or less.

4. The microfluidic chip of claim 1, wherein the nanofiber membrane has an average pore size in a range from about 0.1 μm to about 15 μm.

5. The microfluidic chip of claim 1, wherein the nanofibers have a diameter of about 100 nm or larger.

6. (canceled)

7. (canceled)

8. The microfluidic chip of claim 1, wherein the nanofiber membrane has porosity in a range from about 50% to about 90%.

9. The microfluidic chip of claim 1, wherein the first and second fluid channels are substantially orthogonal to each other.

10. The microfluidic chip of claim 1, wherein the nanofiber membrane separating the first and second fluid channels has an area of about 3 cm² or greater.

11. The microfluidic chip of claim 1, wherein the first and second channel layers comprise polydimethylsiloxane (PDMS).

12. The microfluidic chip of claim 1, wherein the first channel layer comprises at least one access channel extending from a surface of the first channel layer to the first fluid channel and the second channel layer comprises at least one access channel extending from a surface of the second channel layer to the second fluid channel.

13. The microfluidic chip of claim 12, wherein the at least one access channel of the second channel layer aligns with at least one corresponding access channel extending through the first channel layer.

14. A scaffold, comprising:
a scaffold frame comprising a scaffold opening passing through the scaffold frame; and
a nanofiber membrane extending across the scaffold opening, the nanofiber membrane comprising a stack of nanofiber layers, each nanofiber layer comprising nanofibers disposed in a defined direction with a controlled spacing between nanofibers, the nanofibers of each nanofiber layer extending across and cross-linked with nanofibers of an adjacent nanofiber layer.

15. The scaffold of claim 14, wherein the scaffold opening has an area of about 3 cm² or greater.

16. The scaffold of claim 14, wherein the nanofibers have a diameter of about 100 nm or larger.

17. (canceled)

18. The scaffold of claim 14, wherein the nanofibers of each nanofiber layer are substantially orthogonal to the nanofibers of the adjacent nanofiber layer.

19. The scaffold of claim 14, wherein the nanofiber membrane has porosity in a range from about 50% to about 90%.

20. The scaffold of claim 14, wherein the scaffold is incorporated into a transwell insert for insertion in a well or chamber.

21. The scaffold of claim 14, wherein one or both sides of the nanofiber membrane are seeded with one or more cell type.

22. The scaffold of claim 14, wherein one or both sides of the nanofiber membrane are seeded with cell-laden hydrogel or extracellular matrix (ECM).

23. The scaffold of claim 14, wherein the nanofiber membrane is coated with an attachment factor.

24. The scaffold of claim 23, wherein the attachment factor is extracellular matrix (ECM).
